(19) United States
(12) Patent Application Publication
(10) Pub. No.: US 2010/0279961 A1
(43) Pub. Date: Nov. 4, 2010

(54) PHARMACEUTICAL COMPOSITION HAVING A TRIHYDROXY-CHROMENONE DERIVATIVE

(75) Inventors: Lutz Müller-Kuhrt, Potsdam (DE); Christian Mang, Berlin (DE); Michael Thiede, Berlin (DE)

Correspondence Address: MAYER & WILLIAMS PC, 251 NORTH AVENUE WEST, 2ND FLOOR, WESTFIELD, NJ, 07090, US

(73) Assignee: ANALYTICON DISCOVERY GMBH, Potsdam (DE)

(21) Appl. No.: 12/665,879

(22) PCT Filed: Jun. 17, 2008
(86) PCT No.: PCT/DE2008/000984

§ 371 Date: Jun. 2, 2010

(30) Foreign Application Priority Data
Jun. 21, 2007 (DE) 10 2007 029 042.1

(51) Int. Cl. A61K 31/7048; C07H 15/20; C07D 311/74; A61K 31/353; A61P 3/10; A61P 13/12; A61P 9/00; A61P 19/00; A61P 27/02; A61P 29/00; A61P 35/00; A61P 37/00; A61P 17/06

(52) U.S. Cl. 514/27; 536/18.1; 549/400; 514/456

(57) ABSTRACT

The invention relates to a pharmaceutical composition comprising a glucuronate, glucoside, and/or sulfo conjugate of 2-(3,4-dihydroxyphenyl)-3,5,7-trihydroxy-4H-chromen-4-one, or such a conjugate of a derivative of 2-(3,4-dihydroxyphenyl)-3,5,7-trihydroxy-4H-chromen-4-one, or a physiologically well tolerated salt of such a conjugate.

### FIELD OF THE INVENTION

The invention relates to a pharmaceutical composition comprising a conjugate of 2-(3,4-dihydroxyphenyl)-3,5,7-trihydroxy-4H-chromen-4-one, to a method for producing such a pharmaceutical composition and to the uses thereof.

### PRIOR ART AND BACKGROUND OF THE INVENTION

From the documents G. Williamson et al., Free Radical Research 39(5):457-469 (2005), R. L. Prior, Am J Clin Nutr 78 (suppl):570S-578S (2003) and P. A. Kroon et al., Am J Clin Nutr 80:15-20 (2004) it is known in the art that orally administered 2-(3,4-dihydroxyphenyl)-3,5,7-trihydroxy-4H-chromen-4-one or the glycosides thereof are metabolized in the human organism to glucoronate and sulfo conjugates.

From the documents R. L. Prior, J Am Clin Nutr 78 (suppl):570S-578S {2003), P. Castilla et al., Am J Clin Nutr 84:252-262 (2006) and E. Ramiro et al., J Agric Food Chem 53:8506-8511 (2005), it is known in the art that 2-(3,4-dihydroxyphenyl)-3,5,7-trihydroxy-4H-chromen-4-one or the glucoronate or sulfo conjugates thereof have antioxidative, antiinflammatory, antimicrobial, and antitumoral effects.

An oral administration of 2-(3,4-dihydroxyphenyl)-3,5,7-trihydroxy-4H-chromen-4-one has however the drawback that very high dosages have to be used because of the poor bioavailability of these substances and the uncontrolled metabolization of these substances according to individual dispositions.

### TECHNICAL OBJECT OF THE INVENTION

It is therefore the technical object of the invention to provide a pharmaceutical composition, which has an improved bioavailability and reproducible pharmacokinetics, in particular a uniform concentration, compared to 2-(3,4-dihydroxyphenyl)-3,5,7-trihydroxy-4H-chromen-4-one.

### BASICS OF THE INVENTION AND PREFERRED EMBODIMENTS

For achieving the above technical object, the invention teaches a pharmaceutical composition comprising a glucuronate, glucoside and/or sulfo conjugate of 2-(3,4-dihydroxyphenyl)-3,5,7-trihydroxy-4H-chromen-4-one, or such a conjugate of a derivative of 2-(3,4-dihydroxyphenyl)-3,5,7-trihydroxy-4H-chromen-4-one, or a physiologically well-tolerated salt of such a conjugate. A pharmaceutical composition according to the invention can also be employed as a nutritional supplementary agent.

A derivative of 2-(3,4-dihydroxyphenyl)-3,5,7-trihydroxy-4H-chromen-4-one is defined by Formula A, where n and m may be, respectively independently from each other, identical or different, in the range from 0 to 4 (0, 1, 2, 3, 4), and wherein the sum of n+m is always 0, 1, 2, 3, or 4.

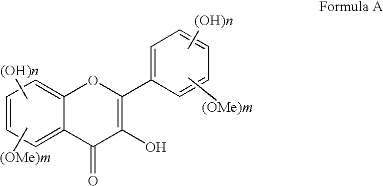

A sulfo conjugate is a derivative of 2-(3,4-dihydroxyphenyl)-3,5,7-trihydroxy-4H-chromen-4-one, where one hydroxyl hydrogen or several hydroxyl hydrogens are replaced by the group —SO₃⁻. A glucuronate conjugate is a derivative of 2-(3,4-dihydroxyphenyl)-3,5,7-trihydroxy-4H-chromen-4-one, where one hydroxyl hydrogen or several hydroxyl hydrogens are replaced by the group of Formula I. The glucuronate conjugates are typically β-D-glucuronates. A glucoside conjugate is a derivative of 2-(3,4-dihydroxyphenyl)-3,5,7-trihydroxy-4H-chromen-4-one, where one hydroxyl hydrogen or several hydroxyl hydrogens are replaced by the group of Formula Ia.

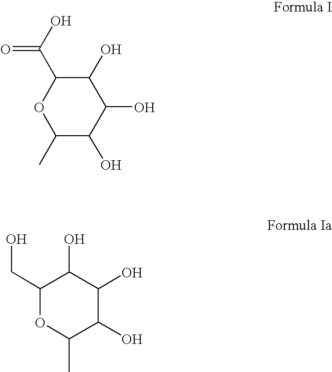

It is also possible that several of the hydroxyl hydrogens may be replaced by several of the above groups. In particular, 1 to 3 groups —SO₃⁻ and/or 1 to 3 groups of Formulas I and/or Ia may be present independently from each other.

The invention is based on the finding that such conjugates have, compared to 2-(3,4-dihydroxyphenyl)-3,5,7-trihydroxy-4H-chromen-4-one or the glycosides thereof, a substantially increased water solubility, so that a pharmaceutical composition may also be supplied to the target tissue as an injection or infusion. Finally, by bypassing the intestinal tract, a reproducible pharmacokinetics will also be achieved, besides specific local administration.

The conjugate may be methylated in one of the positions 3′ or 4′, or in both positions 3′ and 4′. In principle, methylations are however also possible in the positions 3 and/or 5 and/or 7, if applicable in addition to the methylation in the positions 3′ and/or 4′.

The conjugate may carry a glucuronate group, a glucoside group, and/or a sulfate group at one or several of the positions 3, 3′, 4′ and 7. Preferably it is a 3-glucuronate, a 3-sulfate, a 3′-glucuronate, a 3′-sulfate, a 4′-glucuronate, a 4′-sulfate, a 3-glucoside, a 4-glucoside, or a 4′-glucoside.

The conjugate may be selected from the group comprising:
2-(3,4-dihydroxyphenyl)-5,7-dihydroxy-4-oxo-4H-chromen-3-yl hydrogen sulfate;
5,7-dihydroxy-2-(3-hydroxy-4-methoxyphenyl)4-oxo-4H-chromen-3-yl hydrogen sulfate;
5,7-dihydroxy-2-(4-hydroxy-3-methoxyphenyl)4-oxo-4H-chromen-3-yl hydrogen sulfate;
2-(3,4-dimethoxyphenyl)-5,7-dihydroxy-4-oxo-4H-chromen-3-yl hydrogen sulfate;
2-hydroxy-5-(3,5,7-trihydroxy-4-oxo-4H-chromen-2-yl)phenyl hydrogen sulfate;
2-methoxy-5-(3,5,7-trihydroxy-4-oxo-4H-chromen-2-yl)phenyl hydrogen sulfate;
2-hydroxy-4-(3,5,7-trihydroxy-4-oxo-4H-chromen-2-yl)phenyl hydrogen sulfate;
2-methoxy-4-(3,5,7-trihydroxy-4-oxo-4H-chromen-2-yl)phenyl hydrogen sulfate;
2-(3,4-dihydroxyphenyl)-3,5-dihydroxy-4-oxo-4H-chromen-7-yl hydrogen sulfate;
2-(3,4-dimethoxyphenyl)-3,5-dihydroxy-4-oxo-4H-chromen-7-yl hydrogen sulfate;
3,5-dihydroxy-2-{4-hydroxy-3-methoxyphenyl)-4-oxo-4H-chromen-7-yl hydrogen sulfate;
3,5-dihydroxy-2-(3-hydroxy-4-methoxyphenyl)-4-oxo-4H-chromen-7-yl hydrogen sulfate;
6-{[2-(3,4-dihydroxyphenyl)-5,7-dihydroxy-4-oxo-4H-chromen-3-yl]oxy}-3,4,5trihydroxytetrahydro-2H-pyran-2-carbonic acid;
6-{[5,7-dihydroxy-2-(3-hydroxy-4-methoxyphenyl)4-oxo-4H-chromen-3-yl]oxy}-3,4,5-trihydroxytetrahydro-2H-pyran-2-carbonic acid;
6-{[5,7-dihydroxy-2-(4-hydroxy-3-methoxyphenyl)4-oxo-4H-chromen-3-yl]oxy}-3,4,5-trihydroxytetrahydro-2H-pyran-2-carbonic acid;
6-{[2-(3,4-dimethoxyphenyl)-5,7-dihydroxy-4-oxo-4H-chromen-3-yl]oxy}-3,4,5-trihydroxytetrahydro-2H-pyran-2-carbonic acid;
6-{[2-(3,4-dihydroxyphenyl)-3,5-dihydroxy-4-oxo-4H-chromen-7-yl]oxy)-3,4,5-trihydroxytetrahydro-2H-pyran-2-carbonic acid;
6-{[2-(3,4-dimethoxyphenyl)-3,5-dihydroxy-4-oxo-4H-chromen-7-yl]oxy}-3,4,5-trihydroxytetrahydro-2H-pyran-2-carbonic acid;
6-{[2-(3-hydroxy-4-methoxyphenyl)-3,5-dihydroxy-4-oxo-4H-chromen-7-yl]oxy}-3,4,5-trihydroxytetrahydro-2H-pyran-2-carbonic acid;
6-{[2-(3-methoxy-4-hydroxyphenyl)-3,5-dihydroxy-4-oxo-4H-chromen-7-yl]oxy}-3,4,5-trihydroxytetrahydro-2H-pyran-2-carbonic acid;
3,5,7-trihydroxy-2-(3-hydroxy-4-{[3,4,5-trihydroxy-6-(hydroxymethyl)tetrahydro-2H-pyran-2-yl]oxy}phenyl)-4H-chromen-4-one;
3,5,7-trihydroxy-2-(4-hydroxy-3-{[3,4,5-trihydroxy-6-(hydroxymethyl)tetrahydro-2H-pyran-2-yl]oxy}phenyl)-4H-chromen-4-one;
5,7-dihydroxy-3-{[3,4,5-trihydroxy-6-(hydroxymethyl)tetrahydro-2H-pyran-2-yl]oxy}-2-(3,4-dihydroxyphenyl)-4H-chromen-4-one;
3,5,7-trihydroxy-2-(3-hydroxy-4-{[3,4,5-trihydroxy-6-(hydroxymethyl)tetrahydro-2H-pyran-2-yl]oxy}phenyl)4H-chromen-4-one;
3,5-dihydroxy-7-{[3,4,5-trihydroxy-6-(hydroxymethyl)tetrahydro-2H-pyran-2-yl]oxy}-2-(3,4-dihydroxyphenyl)-4H-chromen-4-one,
   and physiologically well tolerated salts of these compounds.

Particularly preferred are 2-(3,4-dihydroxyphenyl)-5,7-dihydroxy-4-oxo-4H-chromen-3-yl hydrogen sulfate (Formula II) and 6-{[2-(3,4-dihydroxyphenyl)-5,7-dihydroxy-4-oxo-4H-chromen-3-yl]oxy}-3,4,5-trihydroxytetrahydro-2H-pyran-2-carbonic acid (Formula III) or salts (e.g. Na+) thereof.

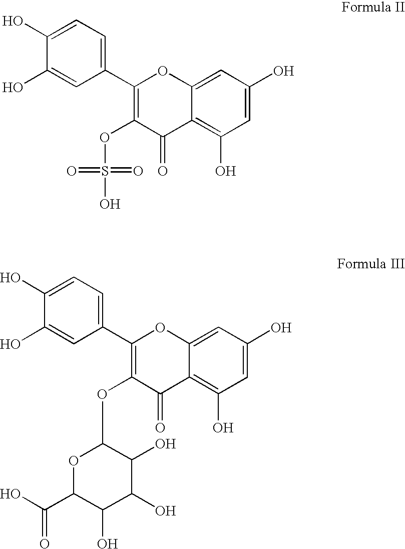

The former can for instance be produced, as described in document D. J. L. Jones et al., Bioorg Med Chem 13:6727-6731 (2005), and by this method with subsequent conventional separation of the different reaction products, other sulfate conjugates than the ones described above can easily be obtained, too. The latter can for instance be produced according to the document M. Boutaib et al., Tetrahedron Lett 43:6263-6266 (2002), and this reaction scheme can easily be adapted to the other glucuronate conjugates.

Because of particularly good water solubility of the substances used according to the invention, it is preferred that the composition is prepared for the parenteral administration, in particular for the intravenous, intra-arterial, intraperitoneal, intra-articular, intraocular, conjunctival, intrapleural, intraventricular, lumbal, intracavitary, or intranasal administration. Typically conventional galenic auxiliary and/or carrier substances are additionally included.

The galenic preparation of the pharmaceutical composition according to the invention may be performed in a conventional manner. As counter ions for ionic compounds may for instance be used Na⁺, K⁺, Li⁺ or cyclohexylammonium or Cl⁻, Br⁻, acetate, trifluoroacetate, propionate, lactate, oxalate, malonate, maleinate, citrate, benzoate, salicylate etc. Suitable liquid galenic forms of preparation are for instance syrups, juices, suspensions, emulsions, drops or injectable solutions (i.v., i.p., i.m., s.c., i.a.) or fine dispersions (aerosols), transdermal systems, and preparations with protracted release of active substance, for the production of which usual means are used, such as solution mediators. As carrier substances are named here in particular solvents, such as sterile water and mono or multi-valent alcohols, for instance glycerin. A pharmaceutical composition according to the invention can be produced by that at least one active substance used according to the invention is mixed in a defined dose with a pharmaceutically suitable and physiologically well tolerated carrier and possibly further suitable active, additional or auxiliary substances in a defined dose, and is prepared in the desired form of administration.

Insofar the invention further relates to a method for producing a pharmaceutical composition according to the invention, wherein a glucuronate, glucoside and/or sulfo conjugate of 2-{3,4-dihydroxyphenyl)-3,5,7-trihydroxy-4H-chromen-4-one, or a physiologically well tolerated salt of such a conjugate is mixed in a physiologically effective dose with galenic auxiliary and/or carrier substances and preferably is prepared for the parenteral administration.

The invention further relates to the use of a substance used according to the invention or of a pharmaceutical composition according to the invention for producing a drug for the treatment and/or prophylaxis of a disease from the group comprising “diabetes mellitus type 2; nephropathy, neuropathy, ophthalmopathy and/or damages to vessels as a consequence of a diabetes mellitus type 1 disease; arterial hypertonia; metabolic syndrome; adipositas; arteriosclerosis; coronary heart diseases; fat metabolic disturbances; consequences of dialysis treatment for terminal kidney insufficient patients; cataract; short gut syndrome; nutritional deficiency symptoms for tumor patients; trauma; viral and bacterial infections, depressions, rheumatoid arthritis; nephritis; Crohn's disease; colitis ulcerosa; pancreatitis; vasculitis; Kawasaki's syndrome; lupus erythematodes; psoriasis; Sjögren's syndrome; Still's syndrome, osteoporosis; plasmozytosis; hyperglobulinemia; multiple myeloma; inflammatory diseases, autoimmune diseases, sepsis; tumor diseases, such as lung cancer, bronchial tumor, leukemia, ovary cancer, sarcomas, meningioma, cancer of the intestine, cancer in the lymph nodes, brain tumor, breast cancer, pancreatic cancer, prostate cancer, bladder cancer, kidney cancer, skin cancer, old age nutritional deficiency symptoms, and as an adjuvant for one or several of the above diseases”. These are diseases, which can be treated or alleviated by active substances with antioxidative, antiinflammatory, antimicrobial, and/or antitumoral effects.

With regard to the USA, the invention also comprises a method for the prophylaxis or treatment of one of the above diseases, wherein a person that suffers from the disease or is in danger of suffering therefrom, is administered a physiologically effective dose of the pharmaceutical composition according to the invention. As daily dose for an adult of 75 kg, a quantity from 10 mg to 10 g, in particular 50 mg to 1 g, should be used. This daily dose may be split up into 1 to 5 individual administrations within a day. The administration of the daily dose may take place on a single day and only once, or several times, for instance daily, every two days, every three days, every four days, every five days, every six days, weekly, every two weeks, every three weeks, or monthly.

In the following, the invention is explained in more detail with reference to embodiments representing examples of execution only.

### Example 1

### Solubilities

In 10 ml water each were dissolved to saturation at 20° C. 2-(3,4-dihydroxyphenyl)-3,5,7-trihydroxy-4H-chromen-4-one (in the following substance 1), K salt of 2-(3,4-dihydroxyphenyl)-5,7-dihydroxy-4-oxo-4H-chromen-3-yl hydrogen sulfate (in the following substance 2), K salt of 6-{[2-(3,4-dihydroxyphenyl)-5,7-dihydroxy-4-oxo-4H-chromen-3-yl]oxy}-3,4,5-trihydroxytetrahydro-2H-pyran-2-carbonic acid (in the following substance 3) and K salt of 5,7-dihydroxy-3-{[3,4,5-trihydroxy-6-(hydroxymethyl)tetrahydro-2H-pyran-2-yl]oxy}-2-(3,4-dihydroxyphenyl)-4H-chromen-4-one (in the following substance 4). For the various substances the dissolved quantities according to Table 1 were found.

TABLE 1

| Substance | Dissolved quantity [mg] |
|---|---|
| 1 | <1 |
| 2 | 96 |
| 3 | 238 |
| 4 | 3.7 |

It can be seen that the substances 2 to 4 used according to the invention can substantially better be dissolved in water than the substance 1, which occurs in plants. Therefore, substances used according to the invention are particularly well suited for formulations for injection or infusion.

### Example 2

### Solution for Infusion or Injection i.v or i.p.

A solution according to the invention for infusion or injection i.v. or i.p. has the following composition:

10 mg water (sterile)
50-250 mg 6-{[2-(3,4-dihydroxyphenyl)-5,7-dihydroxy-4-oxo-4H-chromen-3-yl]oxy}-3,4,5-trihydroxytetrahydro-2H-pyran-2-carbonic acid (Na salt)
0.1-10 mg ascorbic acid (preservation agent, optional).

The water may be replaced by a 0.9% by weight aqueous NaCl solution. The pharmaceutical composition according to the invention may be mixed before the administration, if compatible, with other pharmaceutical compositions, for instance with infusion solutions, electrolytic solutions, lipid solutions, or suspensions or emulsions for artificial nutrition.

1. A pharmaceutical composition comprising a glucuronate, glucoside and/or sulfo conjugate of 2-(3,4-dihydroxyphenyl)-3,5,7-trihydroxy-4H-chromen-4-one, or such a conjugate of a derivative of 2-(3,4-dihydroxyphenyl)-3,5,7-trihydroxy-4H-chromen-4-one, or a physiologically well tolerated salt of such a conjugate.

2. A pharmaceutical composition according to claim 1, wherein the conjugate is methylated in one of the positions 3′ or 4′, or in both positions 3′ and 4′.

3. A pharmaceutical composition according to claim 1, wherein the conjugate carries at one or several of the positions 3, 3′, 4′ and 7 a glucuronate group, a glucoside group and/or a sulfate group, in particular a 3-glucuronate, a 3-sulfate, a 3′-glucuronate, a 3′-sulfate, a 4′-glucuronate, a 4′-sulfate, a 3-glucoside, a 4-glucoside, or a 4′-glucoside.

4. A pharmaceutical composition according to claim 1, wherein the conjugate is selected from the group comprising:
2-(3,4-dihydroxyphenyl)-5,7-dihydroxy-4-oxo-4H-chromen-3-yl hydrogen sulfate;
5,7-dihydroxy-2-(3-hydroxy-4-methoxyphenyl)4-oxo-4H-chromen-3-yl hydrogen sulfate;
5,7-dihydroxy-2-(4-hydroxy-3-methoxyphenyl)4-oxo-4H-chromen-3-yl hydrogen sulfate;
2-(3,4-dimethoxyphenyl)-5,7-dihydroxy-4-oxo-4H-chromen-3-yl hydrogen sulfate;
2-hydroxy-5-(3,5,7-trihydroxy-4-oxo-4H-chromen-2-yl)phenyl hydrogen sulfate;
2-methoxy-5-(3,5,7-trihydroxy-4-oxo-4H-chromen-2-yl)phenyl hydrogen sulfate;
2-hydroxy-4-(3,5,7-trihydroxy-4-oxo-4H-chromen-2-yl)phenyl hydrogen sulfate;
2-methoxy-4-(3,5,7-trihydroxy-4-oxo-4H-chromen-2-yl)phenyl hydrogen sulfate;
2-(3,4-dihydroxyphenyl)-3,5-dihydroxy-4-oxo-4H-chromen-7-yl hydrogen sulfate;
2-(3,4-dimethoxyphenyl)-3,5-dihydroxy-4-oxo-4H-chromen-7-yl hydrogen sulfate;
3,5-dihydroxy-2-{4-hydroxy-3-methoxyphenyl)-4-oxo-4H-chromen-7-yl hydrogen sulfate;
3,5-dihydroxy-2-(3-hydroxy-4-methoxyphenyl)-4-oxo-4H-chromen-7-yl hydrogen sulfate;
6-{[2-(3,4-dihydroxyphenyl)-5,7-dihydroxy-4-oxo-4H-chromen-3-yl]oxy}-3,4,5-trihydroxytetrahydro-2H-pyran-2-carbonic acid;
6-{[5,7-dihydroxy-2-(3-hydroxy-4-methoxyphenyl)4-oxo-4H-chromen-3-yl]oxy}-3,4,5-trihydroxytetrahydro-2H-pyran-2-carbonic acid;
6-{[5,7-dihydroxy-2-(4-hydroxy-3-methoxyphenyl)4-oxo-4H-chromen-3-yl]oxy}-3,4,5-trihydroxytetrahydro-2H-pyran-2-carbonic acid;
6-{[2-(3,4-dimethoxyphenyl)-5,7-dihydroxy-4-oxo-4H-chromen-3-yl]oxy}-3,4,5-trihydroxytetrahydro-2H-pyran-2-carbonic acid;
3,4,5-trihydroxy-6-[2-hydroxy-5-(3,5,7-trihydroxy-4-oxo-4H-chromen-2-yl)phenoxy]tetrahydro-2H-pyran-2-carbonic acid;
3,4,5-trihydroxy-6-[2-methoxy-5-(3,5,7-trihydroxy-4-oxo-4H-chromen-2-yl)phenoxy]tetrahydro-2H-pyran-2-carbonic acid;
3,4,5-trihydroxy-6-[2-hydroxy-4-(3,5,7-trihydroxy-4-oxo-4H-chromen-2-yl)phenoxy]tetrahydro-2H-pyran-2-carbonic acid;
3,4,5-trihydroxy-6-[2-methoxy-4-(3,5,7-trihydroxy-4-oxo-4H-chromen-2-yl)phenoxy]tetrahydro-2H-pyran-2-carbonic acid;
6-{[2-(3,4-dihydroxyphenyl)-3,5-dihydroxy-4-oxo-4H-chromen-7-yl]oxy}-3,4,5-trihydroxytetrahydro-2H-pyran-2-carbonic acid;
6-{[2-(3,4-dimethoxyphenyl)-3,5-dihydroxy-4-oxo-4H-chromen-7-yl]oxy}-3,4,5-trihydroxytetrahydro-2H-pyran-2-carbonic acid;
6-{[2-(3-hydroxy-4-methoxyphenyl)-3,5-dihydroxy-4-oxo-4H-chromen-7-yl]oxy}-3,4,5-trihydroxytetrahydro-2H-pyran-2-carbonic acid;
6-{[2-(3-methoxy-4-hydroxyphenyl)-3,5-dihydroxy-4-oxo-4H-chromen-7-yl]oxy}-3,4,5-trihydroxytetrahydro-2H-pyran-2-carbonic acid;
3,5,7-trihydroxy-2-(3-hydroxy-4-{[3,4,5-trihydroxy-6-(hydroxymethyl)tetrahydro-2H-pyran-2-yl)oxy}phenyl)-4H-chromen-4-one;
3,5,7-trihydroxy-2-(4-hydroxy-3-{[3,4,5-trihydroxy-6-(hydroxymethyl)tetrahydro-2H-pyran-2-yl]oxy}phenyl)-4H-chromen-4-one;
5,7-dihydroxy-3-{[3,4,5-trihydroxy-6-(hydroxymethyl)tetrahydro-2H-pyran-2-yl]oxy}-2-(3,4-dihydroxyphenyl)-4H-chromen-4-one;
3,5,7-trihydroxy-2-(3-hydroxy-4-{[3,4,5-trihydroxy-6-(hydroxymethyl)tetrahydro-2H-pyran-2-yl]oxy}phenyl)4H-chromen-4-one;
3,5-dihydroxy-7-{[3,4,5-trihydroxy-6-(hydroxymethyl)tetrahydro-2H-pyran-2-yl]oxy}-2-(3,4-dihydroxyphenyl)-4H-chromen-4-one,
and physiologically well tolerated salts of these compounds.

5. A pharmaceutical composition according to claim 1, wherein the composition is prepared for the parenteral administration, in particular for the intravenous, intra-arterial, intraperitoneal, intra-articular, intraocular, conjunctival or intranasal administration.

6. A pharmaceutical composition according to claim 1, additionally comprising galenic auxiliary and/or carrier substances.

7. The use of a pharmaceutical composition according to claim 1 for producing a drug for the treatment and/or prophylaxis of a disease from the group comprising “diabetes mellitus type 2; nephropathy, neuropathy, ophthalmopathy and/or damages to vessels as a consequence of a diabetes mellitus type 1 disease; arterial hypertonia; metabolic syndrome; adipositas; arteriosclerosis; coronary heart diseases; fat metabolic disturbances; consequences of dialysis treatment for terminal kidney insufficient patients; cataract; short gut syndrome; nutritional deficiency symptoms for tumor patients; trauma; viral and bacterial infections, depressions, rheumatoid arthritis; nephritis; Crohn's disease; colitis ulcerosa; pancreatitis; vasculitis; Kawasaki's syndrome; lupus erythematodes; psoriasis; Sjögren's syndrome; Still's syndrome, osteoporosis; plasmozytosis; hyperglobulinemia; multiple myeloma; inflammatory diseases, autoimmune diseases, sepsis; tumor diseases, such as lung cancer, bronchial tumor, leukemia, ovary cancer, sarcomas, meningioma, cancer of the intestine, cancer in the lymph nodes, brain tumor, breast cancer, pancreatic cancer, prostate cancer, bladder cancer, kidney cancer, skin cancer, old age nutritional deficiency symptoms, and as an adjuvant for one or several of the above diseases”.

8. A method for the prophylaxis or treatment of a disease according to claim 7, wherein a person that suffers from the disease or is in danger of suffering therefrom, is administered a physiologically effective dose of the pharmaceutical composition according to claim 1.

9. A method for producing a pharmaceutical composition according to claim 1, wherein a glucuronate, glucoside and/or sulfate conjugate of 2-{3,4-dihydroxyphenyl)-3,5,7-trihydroxy-4H-chromen-4-one, or a physiologically well tolerated salt of such a conjugate is mixed in a physiologically effective dose with galenic auxiliary and/or carrier substances and preferably is prepared for the parenteral administration.
